(19) United States
(12) Patent Application Publication
(10) Pub. No.: US 2010/0094430 A1
(43) Pub. Date: Apr. 15, 2010

(54) Device

(76) Inventor: Susan Pran Krumdieck, Christchurch (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/444,958

(22) PCT Filed: Oct. 11, 2007
(86) PCT No.: PCT/NZ07/00303

§ 371 Date: Dec. 24, 2009

(30) Foreign Application Priority Data
Oct. 12, 2006 (NZ) 550531

(51) Int. Cl. A61F 2/28; B05D 3/04

(52) U.S. Cl. 623/23.5; 427/2.27

(57) ABSTRACT

An implant for bone replacement and attachment in an animal's body including, a structural portion having an outer porous surface, a ceramic material applied to the porous surface of the structural portion, characterised in that the thickness of the ceramic material as applied utilizing pulsed pressure MOCVD is such that at least some of the pores of the porous surface are not completely closed.

### TECHNICAL FIELD

This invention relates to a device. More specifically this invention relates to an implant.

### BACKGROUND ART

Orthopedic implants have become of great benefit in recent years. Replacement of a painful and/or dysfunctional joint can eliminate, or at least greatly reduce pain, and also restore some if not all lost function such as walking and general movement. As well as allowing the patient to return to a normal active lifestyle, implants can also reduce a patient's dependence on drugs which can often have negative side effects.

The fact that almost everyone knows someone who has an artificial joint substitute (e.g. finger, hip, knee, not to mention teeth substitutes) illustrates how big the market for bioimplants has become—and it is a growing market. About 500,000 Ti/ceramic hips have been implanted in 1998, with an estimated growth rate of 100,000 per year [Van Sloten et al, 1998]. In Sweden 7% of the total number of hip replacements have been revision operations [http://ww.geocities.com/hip_replacements/statistics.htm. 20 Aug. 2003], a small number compared to the revisions in Australia (13.2%) [http://ww.geocities.com/hip_replacements/statistics.htm. 20 Aug. 2003] and the UK (18%) [Suchanek, Yoshimura, 1998].

90% of joint replacements currently performed are successful for more than 10 years [Van Sloten et al, 1998], but the high proportion of revision surgeries emphasises a need for improvement. Patients would like to benefit from their implants for as long as possible without the risk of secondary surgery. Furthermore, surgeries are an immense cost for the patient as well as for health insurance.

The main problem with attempts to replace damaged tissue in living systems is the natural reaction of the body to destroy any foreign object or—if that is not possible—to encapsulate it in fibrous tissues and separate it from its environment. This makes the fixation of the implant very difficult. Loosening of the implant can lead to increased dynamic loading, and hence fatigue fractures.

Another reason for loosening of the implant is the stress shielding effect. This is the loss of bone that occurs when stress is diverted from the area adjacent to the implant, due to the large difference in stiffness.

These factors have lead to technical and material challenges in long term fixation of orthopaedic bone implants and joint replacements.

The orthopaedic implant can be attached to the bone in several ways.

From the 1960's onwards the most common procedure was to embed the prosthesis stem in a polymeric bone cement, poly(methyl methacrylate) (PMNA) which impregnates the bone and thereby holds the implant to it. Polymeric bone cement is usually used with smooth surfaced implants; it is a brittle material with little resistance to the repeated loads experienced by joints. It also lacks adhesive properties, and therefore acts simply to fill the gaps between the implant and the bone to help the bone support the implant. Motion and rubbing within the joint can result in breakdown of the cement, leading to the implant becoming loose, further pain and the loss of function of the implant. PMNA is adequate for approximately 10 years, but failures are frequent after 15 years. This technique is therefore inadequate for younger patients since revision of the bone cement is difficult.

A newer and more successful method is biological fixation using active surface coatings, first introduced in 1991. These involve the use of implants coated with a porous material, bone grows into the porous surface of the implant, providing a stable bond, which then holds the implant in place. This method overcomes the problems associated with using bone cement; however it also introduces new problems.

The use of porous metal implants for bone replacement and attachment are well-known in the prior art and has been used in surgical implant design (Spector et al 1988) as follows:
1. To fabricate devices to replace or argument soft and hard tissues.
2. As coatings on prosthesis to accommodate tissue and growth for biological fixation.
3. As scaffolds to facilitate the regeneration of tissue.

The purpose of the porous material is to provide a strong and permanent interface between the bone and the implant, by allowing tissue in-growth into the pores of the material which results in a strong interlocking mechanical attachment of the tissue to the porous material.

The porous metal may be made from sintering of metal beads, vapor infiltration deposition or any other method. The metal may be titanium or tantalum, or any other metal containing similar properties. The porous metal-bone interface is in the public domain.

One of the most critical factors for patient recovery is rapid healing of the injured bone surface. The main problem introduced by biological fixation is the initial fixation. The time for bone in-growth into porous implants is approximately eight to twelve weeks. In-growth of bone into the implant relies on a stable connection between implant and the bone without any movement. Therefore partial or complete immobilisation of some joints may be required. The optimal size of porosity for bone in-growth is also known from medical trials.

The revision of implants using biological fixation is very difficult due to the implant being directly connected to the bone. However, due to this same feature, less revisions are required.

Several factors can lead to increased bone deposition by the body into the porous surface of the implant. One is the use of ceramic coatings over the porous implant structure. Ceramic coatings have the advantage that they suffer less from corrosion and can protect the underlying metal. One widely applied coating material is hydroxyapatite (HA) which is a major constituent of bone.

Hydroxyapatite is a biocompatible calcium phosphate (Ca₁₀(PO₄)₆(OH)₂) that crystallises at ˜550° C. and can be found in hard tissues and calcified cartilage. Human bone consists of approximately 43% (weight) HA while the remainder consists of 36% wt collagen and 14% wt water [Biomaterials, introduction]

The structure of HA is almost identical to bone mineral (with a Ca/P ratio of 1.67). If the Ca/P ratio of the hydroxyapatite is lower than 1.67, α- or β-tricalcium phosphate (TCP) forms [Suchanek, Yoshimura, 1998]. The presence of TCP increases slow crack growth susceptibility and biodegradability of the HA ceramics. Higher Ca/P ratio leads to the formation of CaO, which is reported to decrease strength and can furthermore lead to decohesion due to stresses from the formation of Ca(OH)₂ and CaCO and related volume changes [Suchanek, Yoshimura, 1998].

The bio-integrative properties of HA are well known. The material is presently used in bone reconstruction and implantation, its use has been approved by the FDA.

Hydroxyapatite has good osteoconductive properties, which means that it supports bone migration along its surface [LeGeros, 2002].

HA also shows bioactivity. In addition to osteoconduction it creates direct chemical bonds with hard tissues [Park, Bronzino, Biomaterials, Principles and Applications, CRC Press, 2003] and so improves adhesion between coating and bone, by forming apatitelike material or carbonate hydroxyapatite on its surface.

An important advantage of HA over other bioceramics (like alpha-Tricalcium Phosphate (Ca₃(PO₄)₂ or beta-Tricalcium Phosphate (Ca₃(PO₄)₂) is its thermodynamic stability at physiological pH which prevents it dissolving under physiological conditions [http://www.azom.com/details.asp?Article ID=1743#_What_materials_are].

Unfortunately, the fatigue properties of pure HA are very, poor compared to bone. The fracture toughness (K_{IC}) does not exceed 1.0 MPa·m^{1/2}, while the value for bone lies between 2-12 MPa·m^{1/2} [Suchanek, Yoshimura, 1998], [Bronzino, 1995]. Additionally, the Weibull-modulus of HA in wet environments is low (m=5-12) which indicates low reliability of HA implants [Suchanek, Yoshimura, 1998]. Therefore it is not possible to expose HA-implants to high dynamic loadings as experienced in human joints.

However, coating a porous metal implant with HA can significantly improve the bonding between bone and implant. Strong bonding allows efficient stress transfer to the implant so that the mechanical properties of the metal are utilized.

Hydroxyapatite can act to increase the activity of bone deposition. Bone formation occurs via tropocollagen fibres serving as nucleation agents for apatite crystals, the mineral components being withdrawn from the surrounding supersaturated body fluid. The formation of the crystal lattices is initiated within the collagen fibres. They grow until they completely fill and surround the fibres and then provide a surface for the deposition of more hydroxyapatite [Kokubo et al, 2003], [White, Handlerm Smith, 1973].

The bone formation on the hydroxyapatite coating is initiated by the creation of an apatite layer on the HA. This layer forms spontaneously and is a characteristic of bioactive materials, including HA, FA (fluoroapatite, Ca₅(PO₄)₃F) and glass-ceramics.

A chemical bond is then formed between bone and coating to decrease the interfacial energy between them.

Reports that the bioactivity of HA decreases with increasing sintering temperature confirms that the degree of bioactivity can directly be related to the degree of negative charge on its surface. HA sintered at higher temperatures has a smaller number of hydroxyl-ions (OH—) at the surface [Kokubo et al, 2003].

Fluorapatite has the advantage that is more stable at high temperatures than HA [Ciliberto et al, 1997] (melting point at 1630° C. [Agathopoulos et al, 2003]) and shows more activity in the formation of bone-like cells [LeGeros, 2002]; [Sakae et al, 2003]. A comparison of bone formation for coated HA and FA implants showed a clear head start for the FA. Here, the bone formation had already started after 6 weeks, whereas there was no indication for bone formation at this stage for the HA coated implants. The proportion of F has to be controlled, since high contents could cause diseases (e.g. fluorosis) [Sakae et al, 2003].

Several methods have previously been used or proposed to deposit hydroxyapatite onto titanium alloys which are used for porous metal orthopaedic implants. These include plasma spraying, sol-gel, hot isostatic pressing, HVOF, pulsed laser ablation, ion beam sputtering and metal organic chemical vapour deposition (MOCVD). Currently plasma spraying is the only method that is commercially accepted.

A big problem is the mismatch of the thermal expansion coefficient of HA (15 10⁻⁶/° C.) and titanium alloys (8.8 10⁻⁶/° C.). Common coating processes require high temperatures, cooling down leads to different shrinkage behaviour that causes precracks at the interface [Breme et al, 1995]. Attempts to use processes at lower temperatures have not been commercially accepted up to now.

Plasma spraying involves a thermal spraying process where heated and melted particles are propelled towards a substrate where they are flattened and quenched very rapidly.

The success of plasma spraying in industrial applications is mainly due to its simplicity, efficient deposition and comparable low costs [Dong et al, 2003]. During the plasma spraying, the HA has to be maintained at temperatures of about 10,000 K. This generates partial decomposition of the precursor components. The particles experience a rapid cooling rate of approximately 10⁵ K/s [Park et al, 1999] when hitting the surface of the substrate and this leads to various disadvantageous effects:
1. Although HA and Ti are exposed to high temperatures the rapid cooling rate of the HA particles hinders chemical reactions and therefore strong chemical bonds between the HA and the titanium [Park et al, 1999]; [Tsui et al, 1998a]; [Tsui et al, 1998b]; this results in poor adhesion of the HA onto the Ti or other metal.
2. The formation of metastable and amorphous CaP phases is undesirable for three reasons. Firstly, it tends to form a continuous layer that acts as a fracture path [Park et al, 1999]. Secondly, although the bone growth occurs at a faster rate in the presence of an amorphous phase because of the initiation of a fast dissolution [Sun et al, 2001], the readily resorbtion by body fluids leads to a serious weakening of the interface between coating and implant [Park et al, 1999]; [Dong et al, 2003]; [Cheang et al, 1996] as well as the production of particle debris in long term [Sun et al, 2001]. The Food and Drug Administration (FDA) advises a minimum of 62% crystallinity [www.fda.gov, 29/10/2003].
3. Furthermore, natural bone HA found in bone is crystalline, thus the integrity of the bone-implant Interface is compromised [Cheang et al, 1996]. The implant needs to be heat-treated for several hours above the crystallisation temperature (550° C.) to recrystallise the amorphous phase.
4. Pores are formed due to shrinkage and air entrapment and partially unmelted particles [Dong et al, 2003]. Plasma-sprayed coatings therefore tend to have high porosity. It is difficult to achieve the desired pore size of 300-400 μm [LeGeros, 2002]. The higher porosity also makes the HA susceptible to corrosive attacks, since the coating is not dense enough to protect the underlying titanium [Knets et al, 1998].

Although rapid cooling during plasma spraying cannot be avoided, there are options to reduce the disadvantages, such as using graded coatings with varying amounts of Ti.

Of the coating techniques previously utilized, thermal or plasma spraying has been the most commonly used and analysed. This technique has been faced with challenges of producing a controllable resorption response in clinical situations. Besides the set backs, thermally sprayed coatings are continually being improved by using different compositions and post heat treatments which converts amorphous phases to crystalline calcium phosphates.

Other techniques have also been investigated. Techniques that are capable of producing thin coatings include pulsed-laser deposition and sputtering which, like thermal spraying involves high-temperature processing. Other techniques such as electro-deposition, and sol-gel utilise lower temperatures and avoid the challenge associated with the structural instability of hydroxyapatite at elevated temperatures. These however have other significant disadvantages.

The inherent physics of plasma spraying methods as well as other so called “wet” methods lead to the resulting deposits being thick, non adherent and structurally fragile. These factors lead to deposits which can easily and readily crumble, flake or fall off the implant prior to and during implementation.

“Wet” processing methods also lead to thick deposits which can block the pores of the porous material and therefore decrease the efficiency of the biological fixation.

“Wet” processing methods do not penetrate the porous surface matrix and therefore do not lead to good adhesion of either the HA or bone to the metal.

These are all significant disadvantages, and prevent the formation of a thin, consistent and reliable coating which allows for bone in growth and therefore biological fixation.

Advantages and disadvantages of a variety of methods are given in Table 1.

Issues of adhesion to the metal structure and strength of the resulting bone have not been resolved for these methods.

Biomemetic methods to deposit HA on metal implants have also been previously investigated.

Here, the implant gets first soaked in a highly concentrated simulated body fluid solution (SBF). A thin amorphous calcium-phosphate coating is deposited on the metal and then immersed in another SBF-solution with a decreased amount of crystal growth inhibitors. The result is a coating of crystalline calcium-phosphate. Since HA will dissolve over the years the attachment bone/Ti has to be considered. Attempts to make the Titanium surface itself bioactive have been successful.

TABLE 1

| Coating | Deposition | | |
|---|---|---|---|
| Process | Thickness | Advantages | Disadvantages |
| Dip Coating | 0.05-0.5 mm | Inexpensive | Requires high sintering |
| | | Coatings applied | temperatures |
| | | quickly | Thermal expansion mismatch |
| | | Can coat complex | |
| | | substrates | |
| Sputter Coating | 0.02-1 μm | Uniform coating | Line of sight technique |
| | | thickness on flat | Expensive |
| | | substrates | Time consuming |
| | | | Cannot coat complex substrates |
| | | | Produces amorphous coatings |
| Pulsed Laser | 0.05-5 μm | As for sputter coating | As for sputter coating |
| Deposition | | | |
| Hot Pressing | 0.2-2.0 mm | Produces dense | Cannot coat complex substrates |
| and Hot | | coatings | High temperature required |
| Isostatic | | | Thermal expansion mismatch |
| Pressing | | | Elastic property differences |
| | | | Expensive |
| | | | Removal/Interaction of |
| | | | encapsulation material |
| Thermal | 30-200 μm | High deposition rates | Line of sight technique |
| Spraying | | | High temperatures induce |
| | | | decomposition |
| | | | Rapid cooling produces |
| | | | amorphous coatings |
| Sol-Gel | <1 μm Thick | Can coat complex | Some processes require |
| | (using slurry-dip | shapes | controlled atmosphere |
| | coating) | Low processing | processing |
| | | temperatures | Expensive raw materials |
| | | Relatively cheap as | |
| | | coatings are very thin | |
| MOCVD | | Low processing | Conventional methods can be |
| | | temperatures | expensive |
| | | High control of coating | |
| | | characteristics | |
| Biomimetic | ~30 μm | Low temp. process | |
| | | Even deposition | |
| | | possible | |

The governing factor in the longevity of implants is the bone-implant interface and the integrity of the adhesive or joining technique used.

A popular new approach to stabilization of the bone-implant interface is to produce an open scaffold structure at the bone contacting surface of the metal implant. The open structure of the surface allows for blood flow and bone growth into the surface. Titanium and tantalum are bio-compatible metals used for the implant structure.

While these metals have a low rejection rate and low scar tissue growth, they do not stimulate bone growth the way a natural break does.

Current sol-gel and plasma spray methods would not be capable of deposition of HA into porous structures and would block up the holes or pores and therefore prevent the desired in-growth.

One alternative method, which overcomes some of the problems with thermal or plasma spray methods, is metal organic chemical vapour deposition (MOCVD).

During the process of metal organic chemical vapour deposition, precursor gases are delivered into a reaction chamber at approximately ambient temperatures. As they pass over or come into contact with a heated substrate, they react or decompose forming a solid phase which is deposited onto the substrate.

MOCVD provides several advantages that make it a promising process for this kind of coating. The highest temperature reached during the process is about 550° C. [Ciliberto et al, 1997] Thus, creation of an amorphous phase (the main disadvantage of plasma spraying) can be avoided.

Furthermore it is possible with MOCVD to control the deposition process chemically and kinetically. Compared to plasma spraying MOCVD offers improved control over nucleation and growth, deposition rate and final stoichiometry of the coating [Ciliberto et al, 1997].

Thin film ceramics by MOCVD on metal often have very good adhesion (Krumdieck, 2001). There are a limited number of published works describing potential precursors for HA deposition by MOCVD (Allen et al, 1996 and Darr et al, 2004).

The precursors used in those studies were introduced into the reaction chamber by sublimation, which places considerable limitations on the choice of precursor (as they must be sufficiently volatile) and the ability to accurately measure the quantities of precursors that are being introduced under given sets of conditions.

Furthermore, each precursor will require different sublimation conditions and the configuration of the apparatus will must be altered to allow introduction of each additional precursor.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

According to one aspect of the present invention there is provided a device including:

a structural portion having an outer porous surface,
a ceramic material applied to the porous surface of the structural portion,
characterised in that the thickness of the ceramic material as applied is such that at least some of the pores of the porous surface are not completely closed.

According to another aspect of the present invention there is provided a method of producing a device, including:

a structural portion with an outer porous surface,
a ceramic material applied to the porous surface of the structural portion,
including the steps of:
using pulsed-pressure MOCVD to apply the ceramic material such that at least some of the pores of the porous surface are not completely closed.

In a preferred embodiment the device may be an implant, and shall be referred to as such herein.

However, this should not be seen as limiting, as the present invention may also be utilised for any other application where a consistent and reliable thin film of ceramic coating is required on and into a porous surface. These include for example electronic components, optical components, and petrochemical filters to name a few.

In a preferred embodiment the implant may be for bone replacement and attachment in an animal's (human or non-human) body.

In a particularly preferred embodiment the implant may be an orthopaedic implant; this could include artificial joint substitutes, or non joint substitutes.

In a preferred embodiment the structural portion of the implant may be made of metal, and shall be referred to as such herein. This metal may be titanium, tantalum or any other metal suitable for bone replacement and attachment, or any alloy thereof.

One skilled in the art would readily realise that other materials could be utilised as the structural portion for other applications.

The structural portion of the implant may be any existing implant, or any implant designed in the future for bone replacement and attachment.

In a preferred embodiment the outer porous surface of the structural portion may have pore sizes which allow the in-growth of bone to provide strong and permanent interface between the bone and the implant.

Several medical studies have determined the size of pores which allow optimal bone in-growth through ample blood flow. This range is has been reported as being 300-400 microns [LeGeros, 2002].

Therefore, in one preferred embodiment the porous surface of the structural portion may have pore sizes within the range of substantially 300-400 microns.

Because the HA thin film will be just a few microns thick, the presence of the hydroxyapatite film on and throughout the porous surface will not change the blood flow pattern of the implant and will not negatively impact the bone in-growth.

In a preferred embodiment the ceramic material may be a material which has bone-integrated properties.

In a preferred embodiment the ceramic material may be an apatite.

Throughout this specification the term ‘apatite’ should be taken as meaning a compound which has the general formula X₅(YO₄)₃Z, where X is usually Ca²⁺, Y is P⁵⁺ or As⁵⁺, and Z is F⁻, Cl⁻, or (OH)⁻. In preferred embodiments the apatite may have the general formula of Ca₅(PO₄)₃(F,Cl,OH).

In a preferred embodiment the ceramic material may be hydroxyapatite (HA) and shall be referred to as such herein. However this should not be seen as limiting as the ceramic material could also include any other suitable apatite, for example, several recent medical studies have shown that fluoroapatite (Ca₁₀(PO₄)₆F₂) (FA) may be more bioactive than HA [Komlev, et. al, 2004] [Oktar, et. al, 2004].

One concern with using FA would be that the fluorine is absorbed by the body during bone in-growth [Savarino, et. al, 1998]. In the case of the thin-film FA, the increased bio-activity would be realized, but the amount of fluorine would be miniscule because of the small amount of ceramic actually present.

In an alternative embodiment the ceramic material may be bioactive glass.

Bioactive glass may also be used either as filler or as a coating and enhances the osteo-conductivity [Boccaccini et al, 2003], [Ferraz et al, 2001] by providing excellent bio-compatibility at the same time [Suchanek, Yoshimura, 1998]. It is reported that even after short implantation times the glass-coated implants show a clearly higher bone regeneration rate than pure HA-coatings do [Ferraz et al, 2001].

In another alternative embodiment the ceramic material may be a combination of HA and a polymer.

Other biomaterials include HA/polymer composites, that can be produced to suit the mechanical properties of bone (Young's Modulus, fracture toughness, ductility and bioactivity) by adjusting the HA content. Difficulties with processing and toxicity mean they have not been widely accepted yet.

For example in one embodiment the ceramic material may be a HA/collagen composite.

HA/collagen composites are considered to be suitable fillers for large bone replacements due to their excellent osteo-conductivity and controlled biodegradability (slow replacement of the composite by bone).

In some preferred embodiments the ceramic material may also include trace metals to produce materials with higher bioactivity.

In a preferred embodiment the ceramic material may be applied to the porous surface of the structural portion in a thin film in the range of a few microns thickness, which will penetrate into the porous structure with a suitable aspect ratio.

In a preferred embodiment the thin film may be in the range of a few microns to tens of microns thick.

The aspect ratio will depend on the structure of the metal implant, and how far the open pores extend into the matrix. Recent vapour deposited tantalum structures are open through most of the depth. Using the Pulsed-Pressure MOCVD method, the penetration depth can be achieved for different pore sizes and depths by varying the processing parameters, allowing for strong natural bone growth into the metal structure.

In a preferred embodiment the film aspect ratio would be equal to the connected pore depth, that is, the depth which is continuously open via pore pathways to the surface. The aspect ratio is defined as the ratio of the pore opening diameter to the pore depth.

In a preferred embodiment the bone re-growth depth may be equivalent to the depth of ceramic coating into the porous surface of the structural portion of the implant. Preferably bone re-growth depth would be equal to the open pore depth. Bone re-growth to this depth within the porous surface of the structural portion of the implant may allow integration of natural bone structure sufficient to provide a strong interface between the bone and the implant which can withstand the load pressure applied by an active lifestyle.

In a preferred embodiment the film of ceramic material may coat the surface of the pores in such a way that the vast majority of the coated pores are open to the minimum size for in-growth as determined from medical tests.

It should be appreciated that there may be a small percentage of pores which, through the manufacturing process of the metal structure, are only a few microns at the surface. These pores may be closed over by the film. However, they would not have allowed bone in-growth in any case. The thin film of a few microns to tens of microns will not be able to bridge and close up the pores in the desired range of 300-400 microns.

Existing implant products are known to have good bone in-growth and are successful implants.

However, the patient must be immobilized until the in-growth has occurred. This time would be significantly shortened if a HA coating was applied. The manufacturers of these implants recognize this, and they are seeking a means to apply a layer of HA to the outside of the implant.

The surface tension of the “wet method” slurries prevents the material from penetrating the porous structure and results in a crumbly thick deposit which closes up the pores. Plasma spraying on a porous surface would also seal up the surface and produce an un-stable deposit. In addition, plasma spraying is a high temperature process which may alter the structure of the implant. Thus the best mode for depositing HA on a metal implant is to produce a thin film which is adherent on the surface at a relatively low temperature.

The hydroxyapatite chemically stimulates the body to deposit new bone material into its structure. The natural structure of bone is much stronger than hydroxyapatite structure due to the bone being a structured composite material with dense ceramic fibres grown in the directions of greatest stress. Hydroxyapatite is a randomly structured manmade material. While hydroxyapatite chemically stimulates bone growth, the bone growth grows into the existing structure of the hydroxyapatite.

The main advantage of the thin film of hydroxyapatite as produced by the present invention which leaves the majority of the pores of the porous surface open is that it will provide chemical stimulation of bone growth on the surface of the porous metal structure, but will have very little material and thus very little structure. The natural bone will thus grow into the porous material implant structure, establishing its own natural, maximum strength structure.

The thin film into the porous material stimulates natural bone growth into the porous metal thus producing a strong interlocking interface between metal and bone which has a high contact surface area.

The main advantage of this is distributing the load on the bone over a large area and thus reducing the maximum stress in the bone.

A further advantage of the thin film produced by the present invention is that the resulting interlocking structure may also alleviate the stiffness mismatch between metal and bone which can cause bone fatigue and degeneration.

The technology utilizing timed, pulsed injections of a liquid metal-organic precursor solution through an ultrasonic atomizer into a continuously evacuated reactor is public domain and is described in: U.S. Pat. No. 5,451,260. CRF D-1394-Raj, et al. “Method and Apparatus for CVD using Liquid Delivery System with Ultrasonic Nozzle” Sono-Tek Corp. licensee.

This technology commercially available and has been demonstrated to produce thin solid films of ceramic materials from metal-organic liquid precursor solutions.

In a preferred embodiment, the ceramic material may be applied to the porous surface of the structural portion by ‘pulsed pressure metal organic chemical vapour deposition’, or ‘pulsed pressure MOCVD’.

The terminology “Pulsed-Pressure MOCVD” is understood in this patent application to refer to the unique processing method described herein that uses a pulsing reactor pressure with no carrier gas.

The terminology “Pulsed-MOCVD” is found in the literature, where it may mean one of two things:
1. Very rapidly pulsed injection of liquid precursor into a constantly flowing, steady pressure reactor. The deposition mechanisms of this process are exactly the same as for conventional MOCVD. This process was pioneered by Senetaur, in France, and is the subject of a patent owned by a capital equipment company, JIPELEC. The group of Figueras in Spain has recently published some results using this precursor feed method as “Pulsed-MOCVD”.
2. An on-off flow of precursor vapour from a bubbler into a stream of continuous flowing carrier gas at constant pressure. This can be accomplished by alternatively raising and lowering the bubbling frit of the carrier gas into the precursor liquid source. The intermittent precursor supply in a continuous flow can also be realised through solenoid valves. This method produces a “wait time” during deposition which produces more organized crystal structure. This wait time is also produced in the pulsed-pressure MOCVD. One of the prominent groups reporting results using this approach is the group of Funakubo at Tokyo Institute of Technology, Japan.

All other MOCVD and even other methods called Pulsed-MOCVD are constant pressure processes. At constant pressure, the mass transport mode to the surfaces inside the pores is by diffusion from the bulk flow to the solid surface where deposition is consuming the precursor. It is well known that in constant pressure MOCVD, the coating thickness decreases with depth of any surface feature.

In a preferred embodiment the pulsed-pressure MOCVD may use a pulsing reactor pressure with no carrier gas.

This will allow the claimed configuration of thin, solid, adherent film into pores on the porous surface of the implant, such that at least some of the pores are not closed. It also overcomes the disadvantage of many other methods such as the build up of large, powder deposits in the protruding tops of the porous material.

In a preferred embodiment the pulsed pressure operation of the pulsed-MOCVD process will be adjusted for maximum aspect ratio penetration of the metal structure, while depositing only a thin film and leaving at least some of the pores of the porous surface not completely closed.

The operating pressure of the reactor is shown in FIG. 5. The maximum pressure, minimum pressure, and cycle time all play a role in the coverage of three dimensional features. The cycle starts when the reactor is evacuated to the minimum pressure. A particular volume of precursor is injected into the vacuum chamber and flash evaporates to produce the pressure spike. The implant porous structure has been evacuated during the pump-down portion of the pulse cycle, and thus according to the principles of rarefied gas dynamics [Roth, 1976] the gas at higher pressure will fill the space inside the pores as long as the mean free path of the gas is not larger than the pore opening. The maximum pressure of the pulse can be adjusted through adjusting the size of the liquid volume injected so that the mean free path of the vapour molecules is small enough for rapid filling of the pores, what ever size the pores on the particular implant.

The thin film hydroxyapatite film of the present invention will have a much more dense and coherent crystal microstructure than current wet methods or plasma spray methods.

This fine microstructure will lead it to greater adhesion to the metal surface, thereby overcoming the low adhesion of the ceramic material to the porous surface obtained by other methods.

As the ceramic deposition by pulsed pressure MOCVD uses low processing temperatures, this does not affect the integrity of the ceramic material, and overcomes the problems associated with methods involving high temperatures, such as
Adhesion being based mainly on mechanical interlocking;
The formation of meta stable in amorphous calcium phosphate phases;
A highly porous coating due to shrinkage, air entrapment and partially unmelted particles.

Pulsed pressure MOCVD has the unique capability for precise control of both precursor concentration and pressure profile during the deposition pulse cycle. This capability will allow development of a process capable of producing the thin film into pores of a given average size and to a given depth. The exact concentration, maximum and minimum pressure (three processing parameters unique to Pulsed-Pressure MOCVD) will be determined for each particular porous implant structure through experimentation.

The present invention therefore has significant advantages over previous films on porous structures, including the following:
It can provide a consistent thin film throughout the depth of the porous structure,
It is thin enough to allow the pores to remain open throughout the porous surface,
It has strong adhesion, and is not prone to cracking,
When used with bone it stimulates bone growth, through decreasing the time required for bone in-growth into the porous structure, and
The method is undertaken at a low temperature, thus overcoming the high temperature disadvantages mentioned on the previous page.

### BRIEF DESCRIPTION OF DRAWINGS

Further aspects of the present invention will become apparent from the following description which is given by way of example only and with reference to the accompanying drawings in which:

FIG. 1 Shows the structural portion of the implant with a porous surface;

FIG. 2 Shows a schematic of thin film of bio-stimulating ceramic on the porous surface of the structural portion of an implant;

FIG. 3 Shows the “assembly line” processes by which any MOCVD process is accomplished;

FIG. 4 Shows a sequence of processes in pulsed pressure MOCVD;

FIG. 5 Shows the pulsed MOCVD reactor vessel pressure;

FIG. 6 shows the difference between conventional MOCVD and pulsed MOCVD;

FIG. 7a-c Shows the comparison of the deposition kinetics and deposited film thickness between low pressure CVD (7a), normal pressure CVD (7b), and pulsed pressure CVD (7c);

FIG. 8 Shows the control of the pulsed pressure MOCVD process;

FIG. 9 Shows the typical configuration of a metal organic precursor chemical which can be used to make a thin film by pulsed pressure MOCVD;

FIG. 10 Shows a 1 cm² coupon of Titanium with the calcium phosphate thin film.

FIG. 11 Shows a SEM micrograph of the commercial porous tantalum implant produced by Zimmer with the calcium-phosphate thin film applied.

FIG. 12 Shows a higher magnification SEM image of the tantalum scaffolding with the surface conformally coated with the calcium phosphate thin film produced by Pulsed-Pressure MOCVD

FIG. 13 Shows a EDS spectrum of the thin film present on the tantalum scaffold shown in FIG. 11.

FIG. 14a-c Shows morphology of deposited HA film on tantalum scaffold using field emission analytical scanning electron microscope.

FIG. 15a-c Shows a cross section of the deposition from FIG. 14 (15a) and EDS analysis at 0.5 and 4 mm from the surface (15b and c).

### BEST MODES FOR CARRYING OUT THE INVENTION

The present invention provides an improved surface on this structural portion of implants to allow greater adhesion and stronger growth of bone.

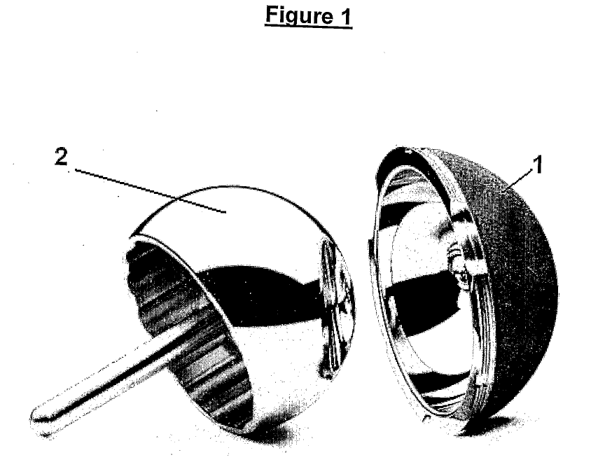

FIG. 1 shows the structural portion of an existing implant, in this example a hip replacement bone implant, both with (1) and without (2) a porous bone integration surface.

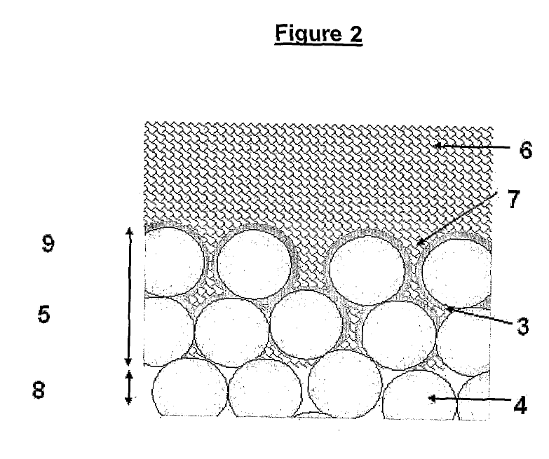

FIG. 2 shows a schematic of the porous surface of the structural portion of the implant. It shows a thin film of hydroxyapatite (3) which has been applied to the porous metal implant structure (4) to the bone re-growth depth (5). The hydroxyapatite coating covers the surface of the pores but leaves at least some of the pores not closed. This provides a porous matrix coated in hydroxyapatite for the original bone (6) to grow (7) into the metal structure. The thin film of the hydroxyapatite allows this growth to be in a natural strong bone structure which increased the strength of the interface between the bone and the implant.

FIG. 2 also shows the average pore size (8) and the film aspect ratio (9).

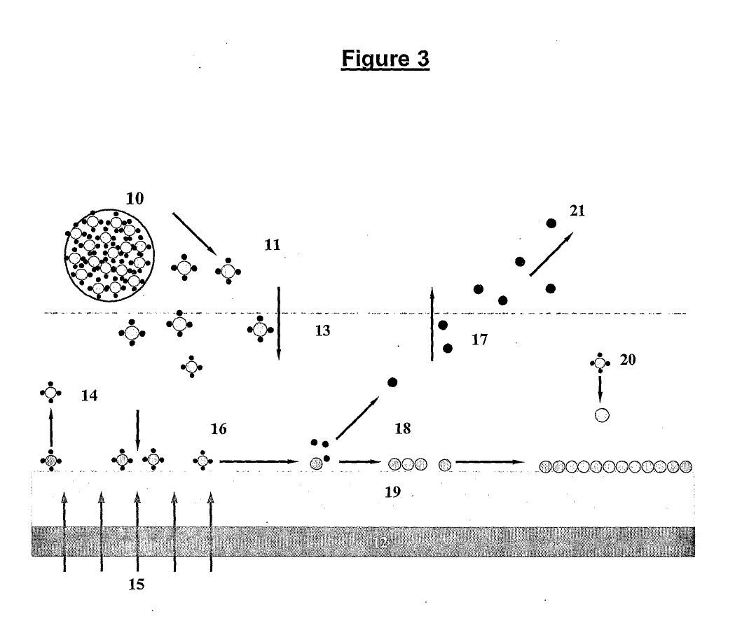

FIG. 3 shows the “assembly line” process by which any kind of MOCVD is accomplished.

The total growth rate of the deposit is controlled by the slowest of all of the processes in the assembly line. In conventional MOCVD, a carrier gas is used to transport a chemical precursor vapor into the zone near the heated substrate. In this situation, the slowest (or rate controlling) step is the diffusion of the precursor vapor from the bulk carrier gas stream through the viscous and concentration boundary layer to the substrate surface where it is consumed. Thus, conventional MOCVD is “diffusion” controlled.

Pulsed-MOCVD achieves process control through direct metering and timed injection of a precise volume of reactant gas into a continuously evacuated reactor. The strategy in running a reactor in this unsteady manner is to achieve relatively high molecular flux rates, uniform film thickness, and minimal impurities. The chemistry of the Pulsed-MOCVD process is the same as the conventional MOCVD process, but the rate limiting process is not the diffusion step, which is usually the case for conventional MOCVD.

In particular reference to FIG. 3; MOCVD is accomplished through an “assembly line” sequence of processes, (10) evaporation of a chemical precursor, (11) mass transport of the precursor vapor to near the substrate (12) surface, (13) diffusion of the precursor to the substrate surface where it is (14) adsorbed and either re-evaporated, or resides long enough to be heated (15) to the reaction temperature (16). The thermal decomposition reaction occurs at a rate dependent on the substrate temperature, k=Aexp−Eₐ/RT), and produces a solid molecule and gas or vapor products (17) which desorb from the surface, are diffused back into the reactor and evacuated from the system (21). Solid molecules on the surface can either (18) nucleate into a new crystal if there is a sufficient number of molecules or (19) be incorporated into a lattice site in an existing crystal according to the well known processes of crystal growth. It is also possible that, if the precursor vapor molecules are radiantly heated enough before encountering the surface, (20) the decomposition can occur in the gas phase, producing a powder particle which can then fall onto the surface or be swept along in the gas flow.

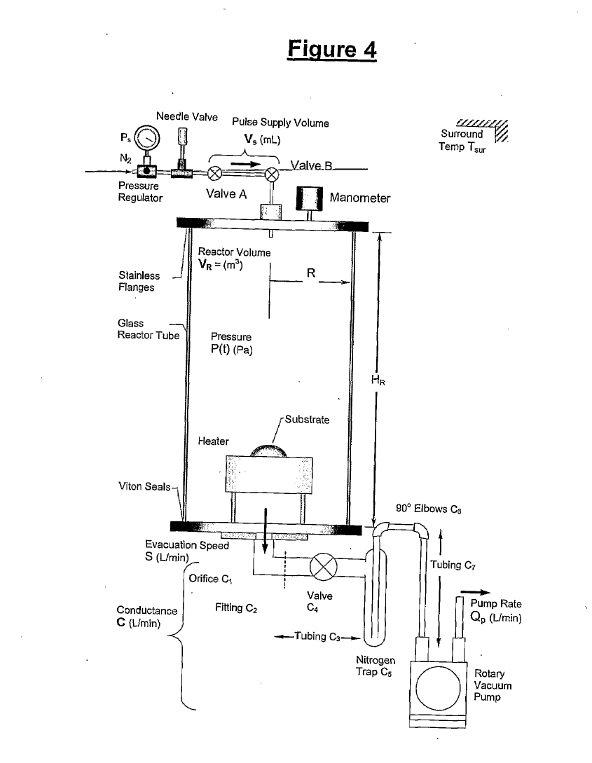

A schematic for a particular experimental Pulsed-Pressure MOCVD system with reactor volume, V_{R}, is shown in FIG. 4. A computer controls the timing of micro solenoid valves to fill the pulse supply volume with gas while valve A is open and B is closed, then inject the gas pulse into the reactor while valve A is closed and B is open. When the gas shot is injected into the reactor at the beginning of each pulse, a pressure spike, Pmax results. Over the balance of the pulse cycle, the reactor is evacuated until the pump-down pressure, Pmin, is reached.

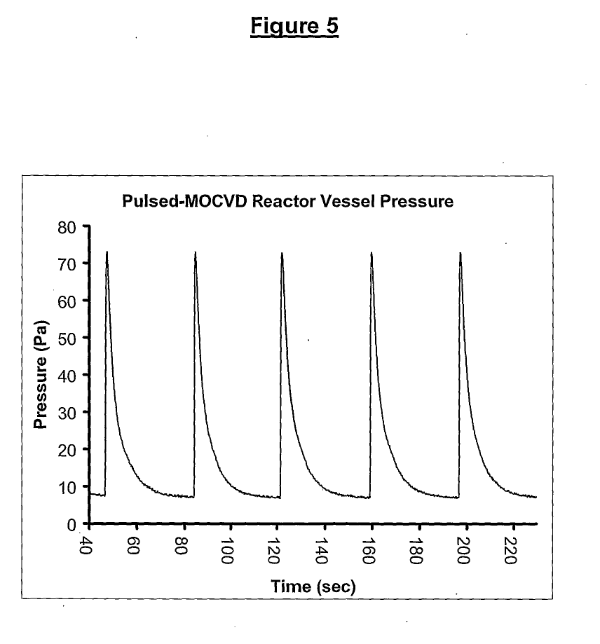

FIG. 5 shows the pressure P(t) in the small reactor over several pulses. Pulse cycle time, t_{P}=38 seconds, reactor volume V_{R}=4.45 liters, pump speed Q_{P}=2.5 liters per second, conductance C=1.64 liters per second, injection volume, V_{S}=1400 mm3, supply pressure, P_{S}=150 Pa(g).

For each pulse, the reactor pressure is given by: [Morosanu 1990]

$${P}^{*} ( t ) = \frac{P ( t ) - {P}_{min}}{{P}_{max} - {P}_{min}} = exp ( - \frac{t}{τ} )$$

where τ is the time constant of the reactor, and Pₘₐₓ is the peak pulse pressure: [Hitchman & Jensen 1993]

$$τ = \frac{{V}_{R}}{S}$$

$${P}_{max} = {P}_{s} ( \frac{{V}_{s}}{{V}_{R}} ) ( \frac{{T}_{R}}{{T}_{s}} ) + {P}_{min}$$

Where the reactor evacuation speed is a function of the pump speed, Q_{P} and the exhaust train conductance, C, S=Q_{P}/C.

### Deposition 3-D Uniformity

The uniformity over a three-dimensional object in the Pulsed-MOCVD process is different than conventional processes, mainly because it is kinetic or mass transport controlled, not diffusion rate controlled.

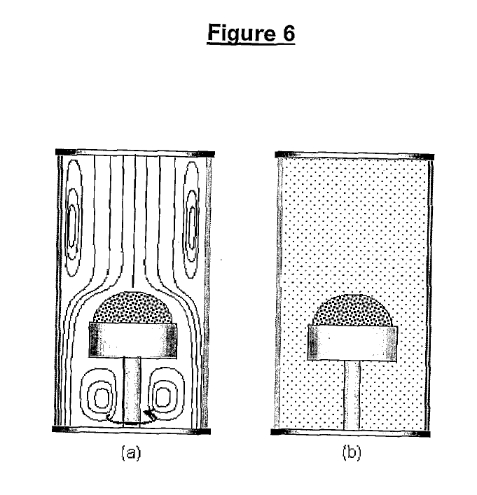

FIG. 6 illustrates the difference between conventional MOCVD and Pulsed-MOCVD, at the same deposition rates; a conventional MOCVD process (a) would take place in the viscous flow range, with the diffusion rate of precursor from the bulk flow to the surface depending on the local boundary layer thickness and bulk flow concentration. In contrast, the Pulsed-MOCVD process (b) has been demonstrated to produce a uniform distribution of precursor throughout the reactor, and thus, the mass transport rate to the surface is uniform over the surface, and is the growth rate controlling step.

The mass transport in Pulsed-MOCVD is accomplished without a carrier gas, eliminating the diffusion process. The capability of Pulsed-MOCVD to coat evenly over complex shapes in three-dimensions is a fundamentally unique aspect at the higher growth rates needed for a product such as the orthopedic implant. High vacuum MOCVD processes are known to have good uniformity, but have very low growth rates and cannot deposit into deep features.

Using the gas dynamics models from rarified gas theory [Roth, 1970] applied to the vapor in Pulsed-MOCVD, we can see that the molecular flux, J(t), to any surface in the reactor at any particular time, t is given by: [Ohring 2002]:

$$J ( t ) = \frac{P ( t )}{\sqrt{2 π {MR}_{o} {T}_{R}}}$$

### Conformality

A key aspect of the innovation of thin-film deposition into porous implants is that the HA coating will extend some depth into the metal structure, but will not close up the openings. MOCVD has been demonstrated to have the capability to produce “conformal” coatings onto step shapes and into holes under certain conditions. Modeling using the Monte-Carlo approach has been done and compared to experiments to show the relationship between deposition parameters and conformal coverage of step shaped holes [Akiyama et al, 2002]. In new research on Chemical Vapor Infiltration (Pulsed-CVI), a pulsed pressure regime has been used to completely fill in the volume of a fiber mat. Pulsed-CVI has produced fully dense carbon-carbon composites [Ohzawa et al, 1999] [Naslain et al, 2001] and polymer fiver bio-implants [Terpstra et al, 2001].

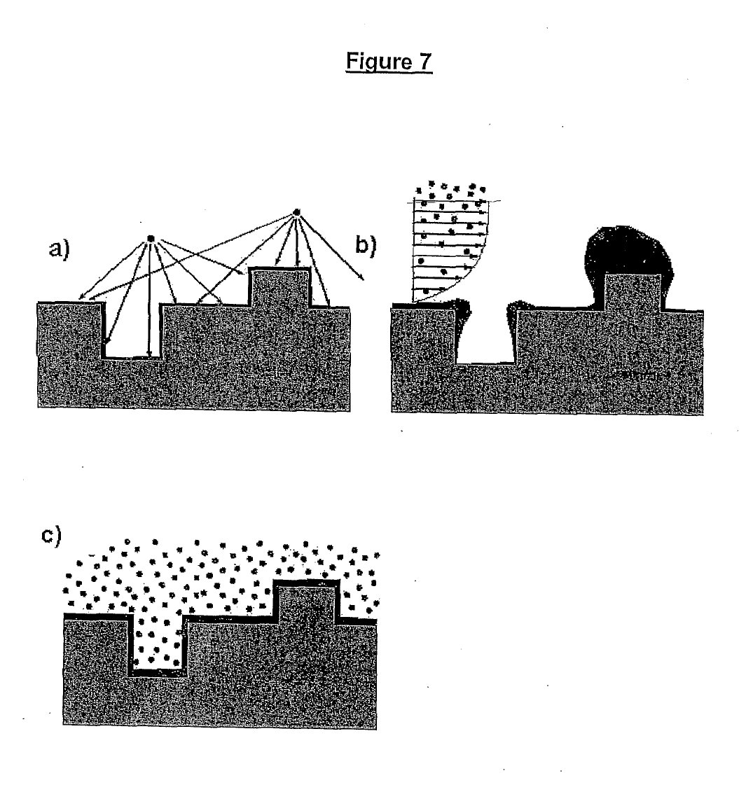

FIG. 7 gives an illustration of the issues of uniform coverage, or conformality, of a thin film deposit on a substrate with three-dimensional surface features. Conformality has been widely studied for conventional CVD processes.

It is well known that low pressure CVD (a) can produce conformal thin films for surface features with aspect ratios (depth compared to opening width) in proportion to the mean free path. In other words, if the mean free path of the low pressure vapor is larger than the opening width, then the probability of molecules penetrating the opening is low, and deposition in the pores will be reduced. It is also well known that higher pressure CVD processes preferentially deposit film on any surfaces protruding up into the bulk gas flow, and on concave surfaces.

Atomic layer deposition (ALD) is a special class of CVD technology which uses intermittent supply of two different reactants. Each reactant is introduced at a partial pressure which allows a mono-layer to form on the substrate surface. ALD has been shown to produce films in holes with very large aspect ratios [Kukli et al] [Gordon et al, 2003]. ALD is done with a continuous carrier gas flow and intermittent precursor introduction into the bulk flow. ALD is usually used to produce very thin films of just a few nanometers.

Pulsed-CVD is a more general technique than ALD, but can be operated in a manner similar to ALD, but with reduced pressure intervals between alternating precursor supply sequences.

The physics of Pulsed-CVD and ALD are similar in that the time and pressure to form a monolayer can be controlled. Thus, the Pulsed-CVD should have the same capability to produce thin films into pores and holes.

FIG. 7b shows atmospheric pressure CVD. In this case, molecular flux rates depend on the relative position of the surface in the boundary layer, the growth rate is high and controlled by the diffusion rate through the carrier gas boundary layer.

FIG. 7c shows pulsed pressure CVD. In this case the molecular flux rate depends on the peak pulse pressure, and is uniform over all surfaces. The precursor is expanded into the reactor without precursor flow, and so fills the evacuated volume uniformly. As the reactor is evacuated after each pulse, the gas diffusivity increases exponentially. Thus over the pulse cycle, the growth rate can be high, and is limited by the integrated partial pressure of the precursor.

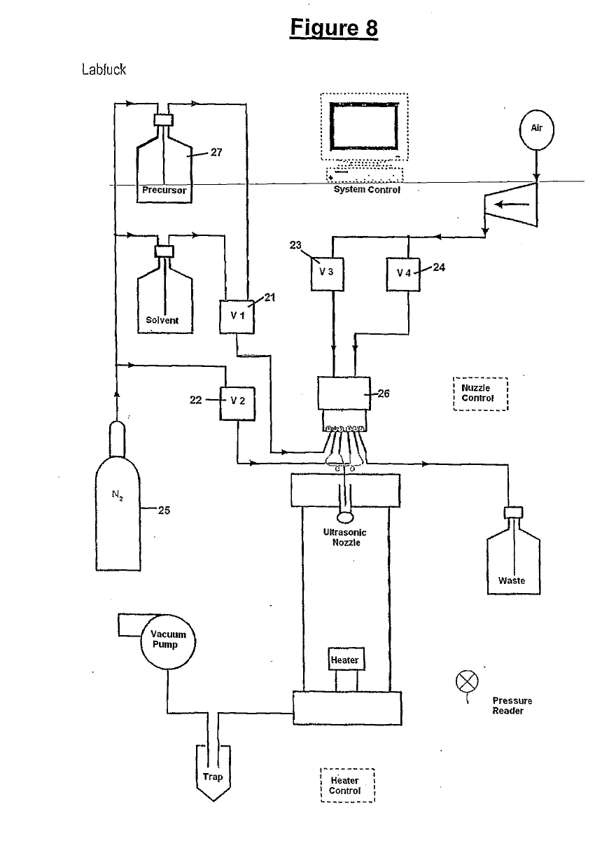

FIG. 8 shows the control of the pulsed pressure MOCVD process.

There are four valves (21, 22, 23 and 24) that are controlled by the control unit. Valve 1 (21) is responsible for the liquid supply (open/closed), while Valve 2 (22) is a 3-way valve and feeds nitrogen from the gas bottle (25) to a filling length L or from there to the system. The NO (normally open) position supplies a filling length L with N₂ while the connection is closed towards the reaction chamber.

The six port external sample injector (26) switches its position by using pressurized air shots either from an open valve 3 (23) (position A), or number 4 (24) opens and turns it back to position B.

Valve 1 (22) is open when the Valco Valve is in Position A (charging, Nr.3 is open). In this position, the sample loop gets filled with liquid precursor (27). Turing Nr.3 off leads to no change in position.

Meanwhile Valve 2 (22) and Nr.4 are closed. Once there is no air left and the sample loop contains only precursor, Nr.1 gets closed.

It is then when the Valco Vale switches to position B (discharge, Nr.4 open) and Nr.2 opens the way from the filling length L to the sample loop and provides the pressure to shoot the liquid in it into the ultrasonic nozzle.

The chemical precursors for MOCVD can be a wide range of thermally decomposed compounds.

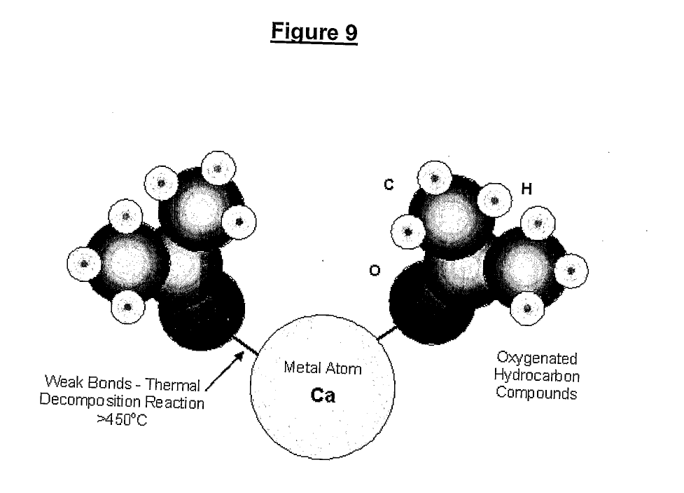

Shown in FIG. 9, is a typical configuration of metal-organic precursor chemical which may be used to make a thin film by pulsed pressure MOVCD.

Both the calcium and the potassium precursor molecules for hydroxyapatite (HA) (Ca₅(PO₄)₃OH), tricalcium phosphate (TCP) (Ca₃(PO₄)) or one of these compounds containing fluorine, consist of the metal atom bound to oxygenated hydrocarbon compounds. A wide range of possibilities exist, and some of the commercially available compounds are listed below:
Ca(C₁₁H₁₉O₂) PO(C₂H₅O)₃
Ca(C₅HF₇O₂)₂ PO(C₃H₇O₂)₃
Ca(C₃H₇O₂)₂ PO(ClCH₂CH₂O)₃

The precursor compound is dissolved into an appropriate solvent for liquid injection into the reactor. Organic solvents are chosen to be compatible with the organic ligands in the precursor, for good vaporization and for good stability and handling. To date, one patent has been issued covering an MOCVD method for Chemical Vapor Infiltration (CVI) of fiber bone implant forms [Senateur et al, 2000]. The patent reviews the CVI process whereby a fiber form is infiltrated and completely filled in and densified with the ceramic HA material.

### Experimentation

The Pulsed-Pressure MOCVD technique has been used to deposit thin films of Calcium Phosphate onto titanium metal coupons and onto tantalum porous bone implants supplied by Zimmer. While optimization of the process is still under research and development, the initial results are included here to illustrate the viability of the claims.

A solution of 0.5 mol % trimethylphosphate and 0.66 mol % Ca[hfpd]₂[triglyme] (where hfpd=1,1,1,5,5,5-hexafluoro-2,4-pentadione) in toluene was prepared. This solution was used as the liquid precursor in the Pulsed-Pressure MOCVD process to deposit Calcium Phosphate on the substrates outlined above.

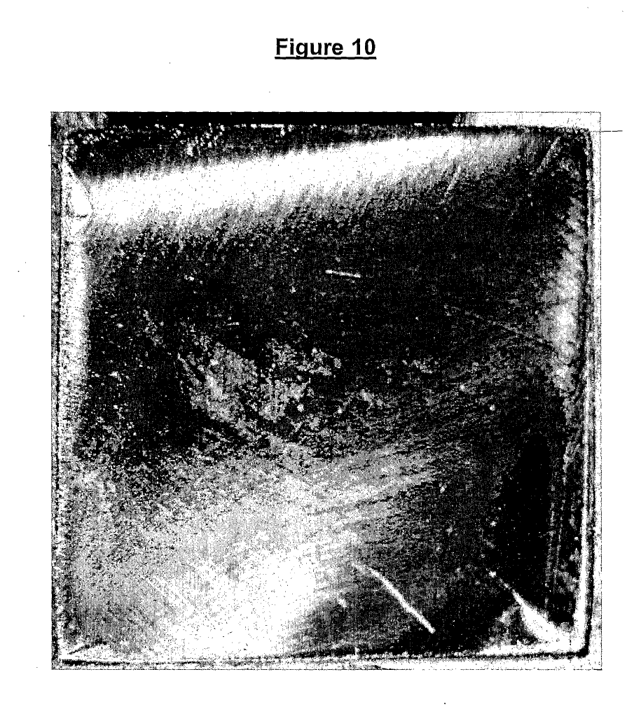

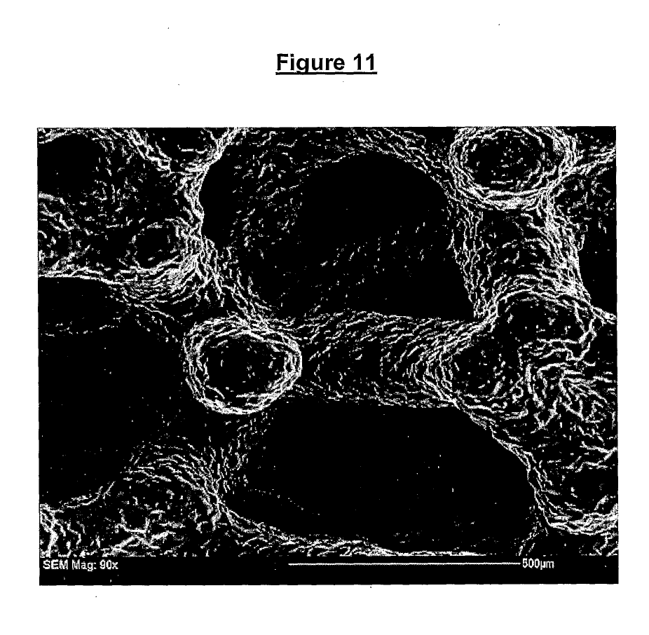

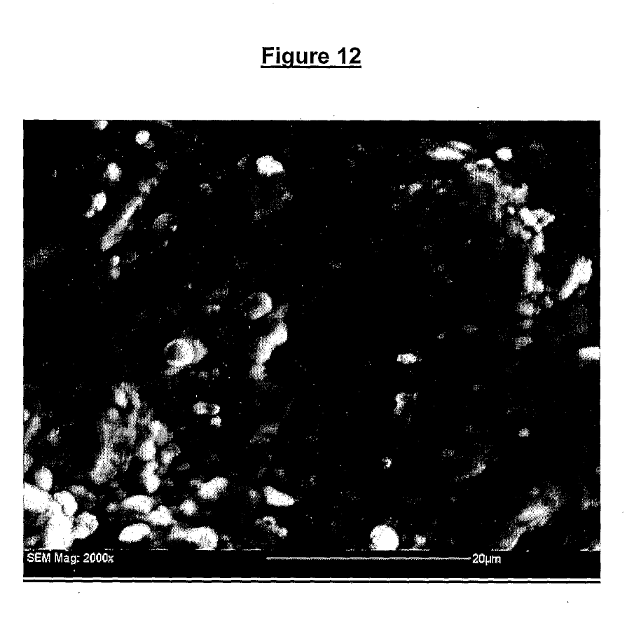

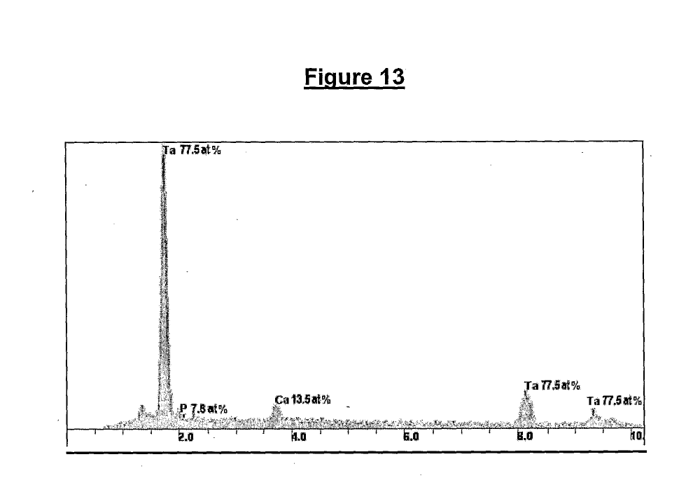

The surface of the deposited films had a flat, glassy appearance as can be seen in FIG. 10 which shows a 1 cm² coupon of Titanium with the calcium phosphate thin film in evidence by the blue colour, and the coloured bands near the holder locations at the upper left and lower right corners. The film is highly adherent, with no cracking, a smooth, uniform surface, which follows the contours of the metal surface. SEM micrographs of the surface of the films deposited on Ti substrates showed little variation from the prepared substrate surfaces with the film appearing to coat conformally over scratches and other topography. The coating on the porous tantalum sample also appeared to provide uniform coverage over the complex surface as shown in FIG. 11 which shows a SEM micrograph of the commercial porous tantalum implant produced by Zimmer with the calcium-phosphate thin film applied. Clearly, the film is not blocking the pores and it is not interfering with the open structure of the implant scaffold. The white arrow marks the location of the EDS analysis shown in FIG. 13. FIG. 12 shows a higher magnification SEM image of the tantalum scaffolding with the surface conformally coated with the calcium phosphate thin film produced by Pulsed-Pressure MOCVD. At the higher magnifications (FIG. 12), the surface appears to be nodular with a limited number of rounded protuberances appearing to grow upwards from the surface.

EDS spectrums collected from the films showed the presence of calcium, phosphorous and titanium/tantalum (FIG. 13). FIG. 13 shows a EDS spectrum of the thin film present on the tantalum scaffold shown in FIG. 11. The presence of the tantalum peak does not indicate that the thin calcium phosphate film does not cover the surface. Rather, the penetration of the x-Ray beam is such that the substrate spectrum are clearly and strongly present in thin film EDS analysis. The Oxygen peak would be off the left hand scale. The ratio of Ca to P is representative of that for HA. A ‘ball park’ estimate of Ca:P ratio can be taken from these EDS results. The Ca:P ratio is an important indicator of which compound in the hydroxyapatite system will form [Suchanek and Yoshimura, 1998]. A stoichiometric ratio of 1.67 is favourable for the formation of hydroxyapatite. At ratios greater than this the formation of CaO is favoured while at ratios lower than this the formation of α- or β-tricalcium phosphate is favored. The Ca:P ratio of films deposited on the porous tantalum samples appeared to vary depending on whether the measurement was taken on raised or low surfaces. The average ratio was found to be 4.0 on raised surfaces of the substrate and 2.4 on lower struts.

The experimental results from these initial investigations compare well with recent results published in the Journal of Biomaterials. [Li et al, 2005] and [Rohanizadeh et al 2005]. However, the pulsed-pressure MOCVD thin films appears to be more of a coherent, uniform coating than a multi-crystalline deposit.

Development of a new precursor system in which calcium and phosphorous ceramic precursors are introduced by liquid injection of a single mixed solution has also been undertaken.

As stated, precursors were chosen that were similar to those that have been used previously for CVD to produce HA or FA. The difference being that the present experimentation involves solution injection as opposed to relying on sublimation or evaporation.

Therefore complete control is possible over precursor ratios by manipulating solution concentration, and no additional bubblers or sublimation chambers are required if additional precursors are to be added to the system (hence our ability to bring in trace metals to produce minerals with higher bioactivity).

There is some limitation in that the precursors must have reasonable solubility in a suitable solvent—to date alcohol has been utilized, but others could be used.

Optimum ratios will be determined empirically.

Use of such a solution means that the quantities and ratios of precursor compounds in the system can be accurately measured and controlled simply by altering the solution composition and measuring the amount that is introduced into the chamber. Additional precursor molecules could also be readily introduced. The objective of this on-going research project is to develop processes to deposit a thin, adherent film of HA deep into a porous tantalum structure without closing the pores.

Details of experimentation undertaken looking at precursor systems is provided below:

### 1. Precursor Development

### 1.1. Materials and Methods

Reagent grade solvents and reagents were purchased from a commercial supplier and were used in the syntheses, solubility and deposition experiments without purification. HPLC grade methanol was used in the precursor solution preparation. ABüchi rotary evaporator equipped with a vacuum pump and water bath (b50° C.) was used to remove solvent from solutions.

### 1.2. Measurements

¹H and ¹³C NMR spectra were recorded on a Varian Unity. 300 Spectrometer with a broadband probe. DMSO-d6 was used as a solvent. Reflectance infra-red spectra were run in KBr powder on a Shimadzu FTIR-8201PC Fourier Transform Infrared Spectrometer. The mass spectrometry experiments were run on a Micromass LCT coupled to a Waters 2790 LC. Scanning electron microscopic analysis was carried out using a JEOL JSM-7000F Field Emission Analytical Scanning Electron Microscope. Using the SEM, energy dispersive X-ray spectros-copy (EDS) data was obtained and element mapping was carried out.

### 1.3. Synthesis

Ca(dbm)2.4H2O dibenzoylmethane (5.01 g, 22 mmol) was dissolved in ethanol (100 mL). The resulting solution was added drop-wise with stirring to Ca(OH)2 (0.76 g, 10 mmol) in a 250 mL beaker. This was left to stir overnight. The compound was filtered and dried in vacuo over fused CaCl2 overnight. Yield 4.20 g (75%). Some of the resulting compound was further purified by extraction with ethanol. Excess ethanol was added to a portion of the compound in a conical flask and was then stoppered and left stirring for two days. After stirring, the solution was filtered directly into a round bottomed flask and the solvent was removed, resulting in compound free of Ca(OH)2. Melting point: 240-244° C. ¹³C NMR: δ 183.2, 141.9, 130.1, 128.2, 127.1, 92.6. ¹H NMR: δ 8.1 (4H), 7.5 (6H), 6.8. IR (KBr) 1596.9, 1519.8, 124 1458.1 cm-1. Calculated for CaC30H22O4. 4H2O: C 64.50, H5.41, N 0. Found C 64.61, H 5.11, N 0.21. TOFMS ES+m/z (%); 225.0897 M+C15H13O2.

### 1.4. Solubility of Precursor Compounds in Methanol

Ca(dbm)2.4H₂O (1 g) was weighed into a 250 mL conical flask. Methanol (50 mL) was added, the flask was stoppered and the solution was swirled for □2 min. The solution was incubated in a water bath at varying temperatures (20°, 30° or 40° C.) for a period of 4 or 17 h. At the end of the incubation period, two 20 mL aliquots of the solution were filtered into separate, pre-weighed round bottomed flasks. All solvent was removed from both samples and the round bottomed flasks were reweighed to determine the mass of compound present in each aliquot and hence the concentration of the saturated solution. Experiments for all conditions were carried out in duplicate. The results of the solubility experiments are compiled in Table 2.

TABLE 2
The solubility (g/100 mL) of
Ca(dbm)2•4H2O in methanol t1:2

| | |
|---|---|
| Incubation | |
| conditions | Ca(dbm)2•4H2O |

| | | |
|---|---|---|
| 20° C. | 4 h | 1.78 ± 0.1 |
| | 17 h | 1.91 ± 0.5 |
| 30° C. | 4 h | 1.86 ± 0.1 |
| | 17 h | 1.88 ± 0.5 |
| 40° C. | 4 h | 1.98 ± 0.2 |
| | 17 h | 1.92 ± 0.4 |

### 1.5. Precursor Solution Preparation

HPLC grade methanol (200 mL) was added to the purified dibenzoylmethane complex (1.95 g, 4 mmol) in a 250 mL graduated laboratory bottle. Trimethyl phosphate (0.28 mL, 2.4 mmol) was then added, the container was sealed and the precursor solution was stirred at room temperature overnight.

### 1.6. Discussion of Precursor Selection and Synthesis

Previous MOCVD studies have used calcium-β-diketonate complexes (Allen et al, 1996, Barr et al, 2004) along with either P2O5 (Allen et al, 1996) or tributylphosphate (Barr et al, 2004) to produce HA coatings. We considered that similar calcium complexes would be appropriate for our initial experiments with the PP-MOCVD technique. Calcium complexes of pentane-2,4-dione (acac), benzoylacetone, and dibenzoylmethane (dbm) were prepared and the dbm complex was chosen for further study because of its higher solubility in methanol. Trimethylphosphate was chosen instead of tributylphosphate because it was available in the laboratory, was also compatible with the methanol solvent, and would not react with the calcium precursor.

The synthesis of Ca(dbm)2.4H₂O was based upon that of Ca (acac)₂, published by Chaudhary et al., except that commercially available Ca(OH)2 was used rather than material prepared from CaCl2. The resulting complex was characterized by melting point, NMR techniques, IR, mass spectrometry, and elemental analysis.

The NMR and mass spectrometric data for the complex was very similar to that for the free dibenzoylmethane, but the high melting point, IR data and elemental analysis provide strong evidence for the formation of the complex. The presence of four water molecules was inferred from the elemental analysis data, and some or all of these are likely coordinated to the calcium ion. We prepared the closely related Ca(acac)2 complex by our method and the data we gathered for the resulting material was identical with that reported in the literature. The solubility experiments were conducted in order to establish the parameters within which the precursor solutions could be prepared for CVD experiments.

### 2. Deposition by Pulsed-Pressure MOCVD

The details of the apparatus and operation of PP-MOCVD have been described elsewhere [Chaudhari et al, 2004). The deposition process in PP-MOCVD consists of repeatable cycles. In each cycle a precise volume of liquid precursor (5 μL) solution is injected through an ultrasonic nozzle into a cold wall reactor chamber. Ultrasonic vibration of the nozzle's tip leads to small liquid droplets formation, which rapidly evaporate in the low pressure (100-600 Pa) inside the chamber. The precursor molecules arrive at the hot substrate, where they are thermally decomposed. The deposition temperature was measured inside the substrate and was fixed at 550° C. The time between pulses was 10 s. Such repeatable changing of the pressure allows the precursors molecules to penetrate deep inside the open structure substrate followed by removal of reaction products and contamination. Total deposition time in this study was 30 min.

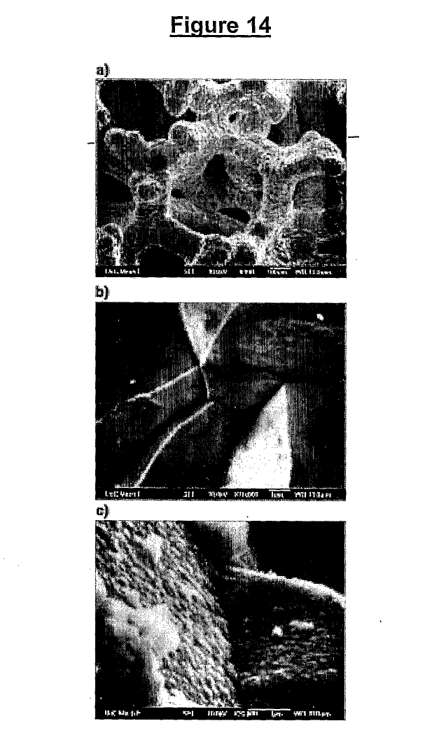

An implant scaffold sample 5 mm thick 10 mm in diameter was cut from a commercially available tantalum knee joint replacement. The sample has open pores which are shown in FIG. 14(a). Prior to deposition, the Ta microstructure is shown in FIG. 14(b). After deposition the sample was again cut and observed in cross section in order to determine morphology of deposited film using field emission analytical scanning electron microscope (JEOLJSM-7000F). Atomic element composition was measured using energy dispersive X-ray spectroscopy (EDS).

### 3. Results

FIG. 14(c) shows the morphology of the HA film deposited on the tantalum scaffold substrate. Compared to the un-coated sample in FIG. 14(b), the ceramic deposits are clearly visible. The ceramic film completely covered all metal surfaces including corners of the Ta grains. This could be observed under SEM by surface charging of non-gold-coated samples. Given the complex shape of the substrate, analysis by XRD was not possible.

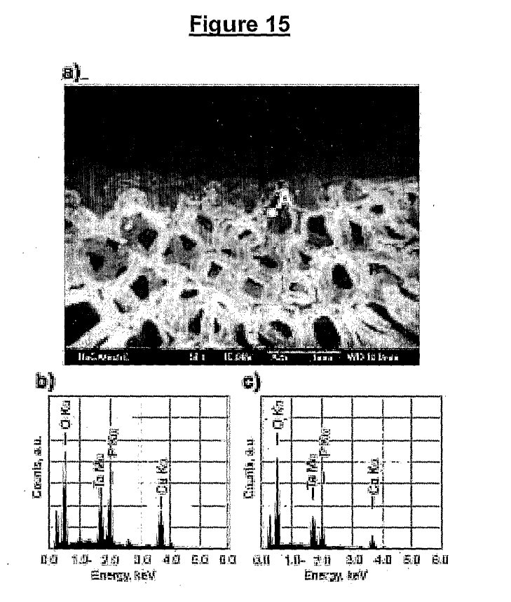

Results of EDS analysis are shown in FIG. 15.

Both calcium and phosphorous are present in the deposited film. At this point it is not clear if the proportions of elemental calcium and phosphorous are indicative of hydroxyapatite Ca₁₀(PO₄)₆(OH)₂ and further experimentation is being carried out to determine the activation energy of both precursor compounds and to determine the solution mixture ratios that yield HA.

The cross section specimens were examined by SEM and EDS to determine the deposition depth into the scaffold structure. The film was observed deep inside Ta foam, at a depth of 0.2 (point A in FIG. 15(a)) and 4 mm as shown in FIGS. 15(b) and (c). It is clear that the thin film deposit is not cracked or spelled, and that it does not close up the scaffold openings.

These initial results point to the suitability of the Ca (dbm)2.4H2O-trimethylphosphate precursor system for MOCVD preparation HA thin films.

A major research effort is now underway to characterize the composition, growth rate, morphology, and bioactive properties of the HA film as a function of precursor ratio and processing parameters; deposition temperature, precursor concentration, and pressure. Future work will include deposition on flat titanium and tantalum substrates to allow more detailed material analysis, process development and cell culture testing of the resulting ceramic. Other calcium complexes will also be prepared and used in MOCVD experiments.

### 4. Conclusion

A new calcium and phosphorous MOCVD precursor system has been developed for hydroxyapatite thin film deposition on tantalum scaffold samples by pulsed-pressure MOCVD. A precursor solution of calcium-dibenzoylmethane and trimethyl-phosphate in methanol was synthesized and analyzed. A precursor solution of 1.95 g, 4 mmol Ca(dbm) complex and 0.28 mL, 2.4 mmol trimethyl phosphate in 200 mL methanol was used to deposit on a substrate Ta scaffold at heater temperature 550° C. Calcium and phosphorous were identified on the tantalum scaffold sample by EDS analysis, and deposition depth was determined to be over 4 mm. SEM analysis confirmed the presence of the ceramic deposits. Work is continuing to determine optimal precursor chemistry and deposition conditions.

Aspects of the present invention have been described by way of example only and it should be appreciated that modifications and additions may be made thereto without departing from the scope thereof.

### REFERENCES

Agathopoulos et al, The fluorapatite-anorthite system in biomedicine, Biomsterials 24, 1317-1331, 2003.
Akiyama, Y, N Imaishi, Y-S Shin, S-C Jung, Macro- and micro-scale simulation of growth rate and composition in MOCVD of yttria-stabilized-zirconia, Journal of Crystal Growth, 241 (2002) 352-262.
Allen et al, Surface and bulk study of calcium phosphate bioceramics obtained by Metal Organic Chemical Vapor Deposition, Nuclear Instruments and Methods in Physics Research, B 116, 457-460, 1996.
http://www.azom.com/details.asp?Article ID=1743#_What_materials_are.
Biomaterials, introduction.
Boccaccini et al, Bioresorbable and bioactive composite materials based on polyactide foams filled with and coated by Bioglass® particles for tissue engineering applications, J. Mat. Science in Medicine 14, 443-450, 2003.
Breme et al, Development of a titanium alloy suitable for an optimized coating with Hydroxyapatite, Biomaterials 16, 239-244, 1995.
Bronzino, The Biomedical Engineering Handbook, CRC Press, IEEE Press, 1995.
Chaudhari, M. K.; Dehury, S. K.; Dhar, S. S.; Bora, U.; Choudary, B. M.; MAnnepalli, L. K. (2004), US Patent Application Number 2004/0127690.
Cheang et al, Addressing processing problems associated with plasma spraying of hydroxyapatite coatings, Biomaterials 17, 537-544, 1996.
Ciliberto et al, Fluorapatite coatings by metal organic chemical vapour deposition, Chemical Communications, 1997.
Darr, J. A.; Guo, Z. X.; Raman, R.; Bououdina, M. (2004). Chem. Commun, Vol. 21, 696.
Dong et al, TEM and STEM analysis on heat-treated and in vitro plasma-sprayed hydroxyapatite/Ti-6Al-4V composite coatings, Biomaterials 24, 97-105, 2003.
Ferraz et al, Histologocial Studies of Double Layer HA/CaO—P205 Glass Plasma Sprayed Coatings using Rabbit Model, Key Engineering Materials Vols. 192-195, 449-452, 2001.
www.fda.gov, 29 Oct. 2003.
http://www.geocities.com/hip_replacements/statistics.htm. 20 Aug. 2003
Gordon, R G, D Hausmann, E Kim, and J Shepart, A kinetic model for step coverage by atomic layer deposition in narrow holes or trenches, Chem Vap Deposition, 9 No. 2 (2003) 73-78.
M L Hitchman and K F Jensen, Chemical Vapor Deposition, Academic Press, London (1993).
Knets et al, Glass and hydroxyapatite coating on titanium implant, 11^{th} conference of the ESB, Toulouse, France, Jul. 8-11 1998.
Kokubo, Kim, Kawashita, Novel bioactive materials with different mechanical properties, Biomaterials 24, 2161-2175, 2003
Komlev, Barinov, Girardin, Oscarsson, Rosengren, Rustichelli, Orlovskii, Porous spherical Hydroxyapatite and fluorhydroxyapatite granules: Processing and characterization, Science and Technolog of Advanced Materials, 2004.
Kukli, K., M. Ritala and M. Leskelä, Atomic layer deposition and chemical vapour deposition of tantalum oxide by successive and simultaneous pulsing of tantalum ethoxide and tantalum chloride.
Krumdieck, S. P, PhD Dissertation, Univ of CO at Boulder (1999).
Krumdieck, S. P.; Acta Mater., 49 (2001) 583-588.
Krumdieck, S. P,; Raj, R. (2001) Adv. Mat. Chem. Vap. Dep., Vol 7 (No. 2), 85.
LeGeros, Properties of Osteoconductive Biomaterials: Calcium Phosphates, Clinical Orthopaedics and Related Research 395, 81-98, 2002.
Li, L-H, H-W Kim, S-H Lee, Y-M Kong, H-E Kim, Biocompatibility of titanium implants modified by microarc oxidation and hydroxyapatite coating, Journal of Biomedical Materials Research Part A, Vol. 73A, Issue 1, 48-54, 2005.
C E Morosanu, Thin Films by Chemical Vapor Deposition, Elsevier, Amsterdam, (1990).
Naslain, R R, R Pailler, X Bourrat, S Bertrand, F Heurtevent, P Dupel and F Lamouroux, Synthesis of highly tailored ceramic matrix composites by pressure-pulsed CVI, Solid State Ionics, 141-142 (2001) 541-548.
M Ohring, Materials Science of Thin Films, Academic Press, San Diego (2002).
Oktar, Ozsoy, Ozygin, A radiological follow-up study of plasma sprayed fluorapatite coatings, Key Engineering Materials, Vol 240-242.
Y Ohzawa, T Sakurai, and K Sugiyama, Preparation of a fibrous SiC shape using pressure-pulsed chemical vapour infiltration and its properties as a high-temperature filter, Journal of Materials Processing Technology, 96 (1999) 151-156.
Park, Bronzino, Biomaterials, Principles and Applications, CRC Press, 2003
Park et al, Graded coating of hydroxyapatite and titanium by atmospheric plasma spraying, Mat. Letters 40, 228-234, 1999.
Rohanizadeh R., R. Z. LeGeros, M. Harsono, A. Bendavid, Adherent apatite coating on titanium substrate using chemical deposition, Journal of Biomedical Materials Research Part A, Vol. 72A, Issue 4, 428-438, 2005.
Roth, A. Vacuum Technology, North Holland, Amsterdam, 1970/1976/1990
Sakae et al, Bone Formation induced by Several Carbonate-and Fluoride-Containing Apatite implanted in Dog Mandible, Key Eng. Mat. 240-242, 395-398, 2003.
Savarino, Stea, Ciapetti, Granchi, Donati, Cervellati, Visentin, Moroni, Pizzoferrato, The interface of bone microstructure and an innovative coating: an X-ray diffraction study, J. Biomed. Mater. Res, 40, 86-91, 1998.
Senateur, J-P., C. Dubourdieu, F. Weiss, M Rosina and A Arbutis, Pulsed injection MOCVD of functional electronic oxides, Advanced Materials for Optics and Electronics, 10 (2000) 155-161.
Sheugraf, K. K., Handbook of Thin-Film Deposition Processes and Techniques, Noyes Publications, New Jersey, 1988.
Spector et al Porous materials. In: Webster J G (ed) Encyclopedia of medical devices and instrumentation, Vol 1. A Wiley-interscience publication John Wiley & Sons, New York, 2335-2341, 1988.
Suchanek, Yoshimura, Processing and properties of hydroxyapatite-based biomaterials for use as hard tissue replacement implants, J. Mater. Res., Vol. 13, No. 1, January 1998.
Sun et al, Material Fundamentals and Clinical Performance of Plasma-Sprayed Hydroxyapatite Coatings: A Review, J. Biomed. Mat. Res (Appl. Biomat) 58, 570-592, 2001.
R A Terpstra, Y G Roman, K Timmer, H A Meinema, Method for manufacturing a fiber-reinforced bioactive ceramic implant, Nederlandse Organisatie Voor Toegepast-Natuurwetenschappelijk Ondersoek, Assignee, U.S. Pat. No. 6,248,392, Jun. 19, 2001.
Tsui et al, Plasma sprayed hydroxyapatite coatings on titanium substrates Part 1 Mechanical properties and residual stress levels, Biomaterials 19, 2015-2029, 1998;
Tsui et al, Plasma sprayed hydroxyapatite coatings on titanium substrates Part 2: Optimisation of coating properties, Biomaterials 19, 2031-2043, 1998;
Van Sloten et al, Materials selection and design for orthopaedic implants with improved long-term performance, Biomaterials 19, 1455-1459, 1998.
White, Handlerm Smith, Principles of Biochemistry, 5th Edition, McGraw Hill, 1973.

1. An implant for bone replacement and attachment in an animal's body including,
a structural portion having an outer porous surface,
a ceramic material applied to the porous surface of the structural portion,
characterised in that the thickness of the ceramic material as applied utilizing pulsed pressure MOCVD is such that at least some of the pores of the porous surface are not completely closed.

2. A device as claimed in claim 1 wherein the device is an orthopedic implant.

3. A device as claimed in claim 1 wherein the structural portion is metal.

4. A device as claimed in claim 3 wherein the structural portion is titanium or tantalum.

5. A device as claimed in claim 2 wherein the porous surface of the structural portion has pore sizes which allows the in growth of bone.

6. A device as claimed in claim 5 wherein the pore sizes of the porous surface is within the range of substantially 300 to 400 microns.

7. A device as claimed in claim 6 wherein the thickness of the ceramic material is within the range of substantially a few to tens of microns.

8. A device as claimed in claim 2 wherein the ceramic material has bone integration properties.

9. A device as claimed in claim 2 wherein the ceramic material is an apatite.

10. A device as claimed in claim 2 wherein the ceramic material is hydroxyapatite.

11. A device as claimed in claim 3 wherein the ceramic material is bioactive glass.

12. A device as claimed in claim 2 wherein the ceramic material is an apatite and polymer composite.

13. A device as claimed in claim 12 wherein the ceramic material is a hydroxyapatite/collegan composite.

14. A device as claimed in claim 9 wherein the ceramic material includes trace metals.

15. A device as claimed in claim 5 wherein the ceramic material is applied to the surface of the porous surface to the connected pore depth.

16. A method of producing a device as claimed in claim 1, including
a structural portion with an outer porous surface,
a ceramic material applied to the porous surface of the structural portion,
including the steps of using pulsed pressure MOCVD to apply the ceramic material such that at least some of the pores of the porous surface are not completely closed.

17. A method as claimed in claim 16 wherein the pulsed pressure MOCVD uses a pulsing reactor pressure with no carrier gas.

18. A method as claimed in claim 16 wherein the pressure is pulsed between a minimum and maximum of substantially 5 and 75 Pa.

19. (canceled)

20. (canceled)
